(19) United States
(12) Patent Application Publication
(10) Pub. No.: US 2021/0372551 A1
(43) Pub. Date: Dec. 2, 2021

(54) TUBING SYSTEM WITH REASSEMBLY PREVENTION MECHANISM

(71) Applicant: Linear Health Sciences, LLC, Oklahoma City, OK (US)

(72) Inventors: Daniel Lawrence Clark, Manhattan Beach, CA (US); Ryan Wayne Dennis, Oklahoma City, OK (US); John Clifford Hayes, Morrisville, NC (US); Stephen James Couse, Morrisville, NC (US)

(21) Appl. No.: 17/335,060

(22) Filed: May 31, 2021

Related U.S. Application Data
(60) Provisional application No. 63/032,609, filed on May 30, 2020.

(51) Int. Cl. F16L 37/367; F16L 37/091; A61M 39/10

(52) U.S. Cl. CPC F16L 37/367; A61M 2039/1077; A61M 39/1011; F16L 37/091

(57) ABSTRACT

A tubing connector system is configured to connect first and second pieces of medical tubing. The tubing connector system includes a male module and a female module. The female module is retained in connection to the male module by a plurality of locking mechanisms. The plurality of locking mechanisms are configured to prevent the male module and female module from being reconnected after the male module has been separated from the female module. The tubing connector system also includes a valve assembly that includes a first valve member contained within the male module and a second valve member contained within the female module. The first and second valve members are interconnected in a gimbaled relationship.

### RELATED APPLICATIONS

The present application claims the benefit of U.S. Provisional Patent Application Ser. No. 63/032,609 filed May 30, 2020 entitled, “Tubing System with Reassembly Prevention Mechanism,” the disclosure of which is herein incorporated by reference.

### FIELD OF THE INVENTION

The present invention is related to medical access devices, and more particularly to a disconnect system for medical tubing.

### BACKGROUND OF THE INVENTION

Medical access devices are used in the treatment of hospitalized patients for a variety of purposes, including intravenous catheters, feeding tubes, Foley catheters, chest tubes, and a variety of surgical drains. Many of these medical access devices transport fluids to or from the patient and use a variety of flexible tubes to give the patient a range of movement during treatment. Unfortunately, due to the freedom of movement that some patients exhibit, the tubing associated with medical access devices is often subjected to forces that cause damage to the tubing, the patient, or both. For example, the tubing typically used in the administration of intravenous fluids is often several feet long, and accordingly can become entangled on hospital beds or other medical equipment surrounding the patient. As the patient moves, the tubing can be stretched and disconnected. In extreme cases, the fluids being administered to the patient, or the patient's own body fluids can be spilled, creating a risk of contamination to the patient's treatment environment, and potentially exposing the patient to a risk of infection.

To alleviate these concerns, a number of different tubing connectors and adapters have been developed that are designed to “break away” when subjected to sufficient tensional forces. In some cases, these connectors include internal valves that prevent fluid from passing through the separated connector. Although these products are often effective at mitigating leaks from the separated adapter, these prior art connectors present a contamination risk if the patient or caregiver attempts to reconnect the tubing adapter. Once the tubing adapter has been disconnected and exposed to the non-sterile environment, reassembly of the adapter presents a significant contamination risk that may increase the probabilities for patient infection. Accordingly, there is a need for an improved tubing adapter that presents the benefits of a leak-resistant breakaway design, while also mitigating the contamination and infection risks associated with the reconnection of the separated adapter. It is to these and other deficiencies in the prior art that the present embodiments are directed.

### BRIEF DESCRIPTION OF THE DRAWINGS

FIG. 1 presents a perspective view of a tubing connector system constructed in accordance with an exemplary embodiment.

FIG. 2 presents an isolated perspective view of the breakaway assembly of the tubing connector system of FIG. 1.

FIG. 3 presents a perspective view of the tubing connector system of FIG. 1 in which the breakaway assembly has been disconnected.

FIG. 4 presents an isolated view of the disconnected breakaway assembly of the tubing connector system of FIG. 3.

FIG. 5 presents an end view of the tubing connector system of FIG. 1.

FIG. 6 presents a side cross-sectional view of the tubing connector system of FIG. 5.

FIG. 7 presents an end view of the tubing connector system of FIG. 1.

FIG. 8 presents a side cross-sectional view of the tubing connector system of FIG. 7.

FIGS. 9A-9C present isolated views of the valve assembly from the tubing connector system of FIG. 1.

FIG. 10 presents an end view of the isolated breakaway assembly in a connected state.

FIG. 11 presents a side cross-sectional view of the isolated breakaway of FIG. 10.

FIG. 12 presents an end view of the isolated breakaway assembly in a disconnected state.

FIG. 13 presents a side cross-sectional view of the isolated breakaway of FIG. 12.

FIG. 14 presents an end view of the isolated breakaway assembly in a disconnected state.

FIG. 15 presents a side cross-sectional view of the isolated breakaway of FIG. 14.

FIG. 16 presents a close-up cross-sectional view of the engagement between the locking collar and the locking ring in a connected state.

FIG. 17 presents a close-up cross-sectional view of the engagement between the locking collar and the locking ring in a disconnected state.

FIG. 18 presents a close-up view of the engagement between stabilizer, alignment tab and locking collar.

FIG. 19 presents a perspective view of the engagement between the female module and the assembly tool.

FIG. 20 presents a close-up perspective view of the assembly tool.

FIG. 21 presents an end view of the engagement between the assembly tool, the female module and the male module.

FIG. 22 presents a side cross-sectional view of the engagement between the assembly tool, the female module and the male module.

FIG. 23 presents a close-up cross-sectional view of the engagement of the wedges of the assembly tool with the locking tabs of the locking collar.

### WRITTEN DESCRIPTION

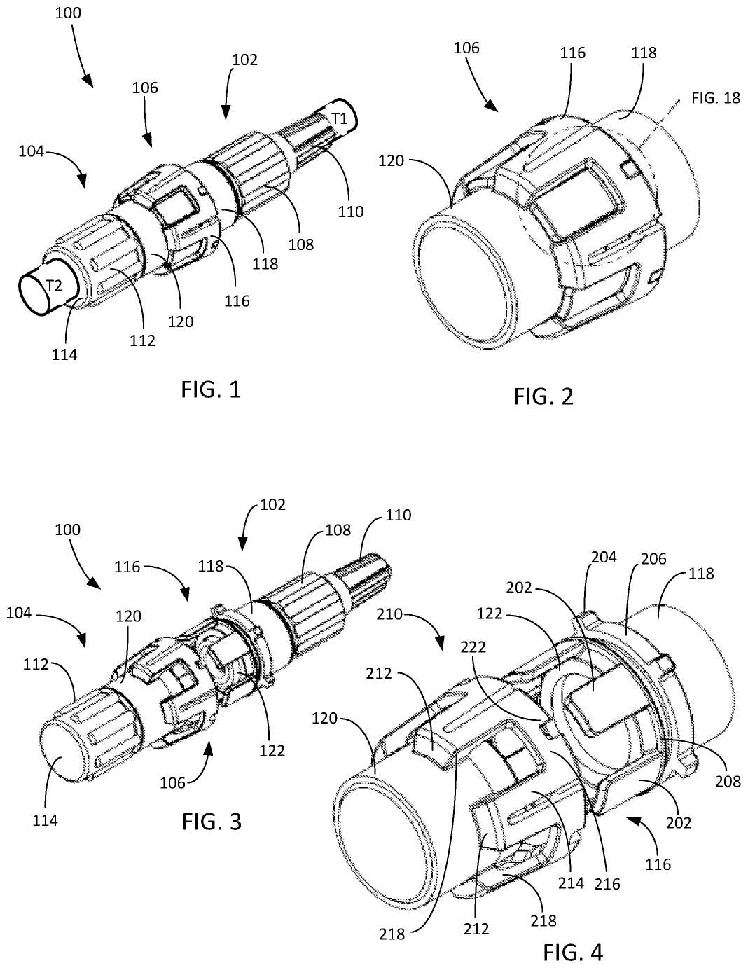

FIG. 1 shows an embodiment of a tubing connector system 100 that is configured to connect two pieces of medical tubing. The tubing connector system 100 includes a first tubing adapter 102, a second tubing adapter 104, and a central connector 106 between the first and second tubing adapters 102, 104. The tubing connector system 100 is generally designed to be used as a single-use, breakaway mechanism between two lengths of medical tubing that are connected between a patient and either an upstream fluid source (e.g., an IV bag) or downstream fluid container (e.g., a drain or Foley catheter). It will be appreciated that the tubing connector system 100 is particularly well suited for use in connecting an upstream bag of medicinal solution to a patient through an intravenous line. In some embodiments, the first and second tubing adapters 102, 104 are attached to the central connector 106 in a manner that permits the rotation of the central connector 106 with respect the first and second tubing adapters 102, 104.

As used in this disclosure, it will be appreciated that tubing connector system 100 is generally cylindrical and symmetrical about a longitudinal axis that extends through the middle of the central connector 102 between the first and second tubing adapters 102, 104. A reference to “longitudinal” will refer to a direction or axis that is parallel or co-linear with the central longitudinal axis extending through the tubing connector system 100. A reference to a radial direction or radial axis will be understood to be in a direction that is substantially orthogonal to the central longitudinal axis. A reference to a rotational movement or direction will be understood to be a reference to a clockwise or counterclockwise movement around the longitudinal axis (unless another axis or rotation is specified). When describing features within the tubing connector system 100, a reference to an interior feature or inward direction refers to something toward the middle of the central connector 106 (either radially or longitudinally), while an exterior feature or outward direction refers to something away from the center of the tubing connector system 100 (again either radially or longitudinally). Unless otherwise noted, the components of the tubing connector system 100 are manufactured from medical grade plastic that is easily sterilized during manufacture.

The first tubing adapter 102 is configured for connection to the first piece of the medical tubing (T1). The second tubing adapter 104 is configured for connection to the second piece of the medical tubing (T2). As depicted, the first tubing adapter 102 includes a first compression fitting 108 that retains a first tubing fitting 110 in connection with the central connector 102. Similarly, the second tubing adapter 104 includes a second compression fitting 112 that retains a second tubing fitting 114 in connection with the central connector 106. In some embodiments, the first and second tubing adapters 102, 104 are configured for use in connecting conventional “Luer” adapters that rely on a friction-fit connection with the medical tubing. It will be appreciated that the first and second tubing adapters 102, 104 can be configured to connect a variety of medical tubing fittings to the central connector 106.

The central connector 106 includes a two-part breakaway assembly 116, depicted in isolation in FIG. 2, that includes a male module 118 connected to a female module 120 in a manner that permits the male module 118 and female module 120 to be separated under the application of sufficient tensile force. The separated central connector 106 is depicted within the tubing connector system 100 in FIG. 3, with the separated breakaway assembly 116 of the central connector 106 shown in isolation in FIG. 4. The central connector 106 retains a two-part valve assembly 122 that includes a first valve member 124 within the male module 118 and a second valve member 126 within the female module 120.

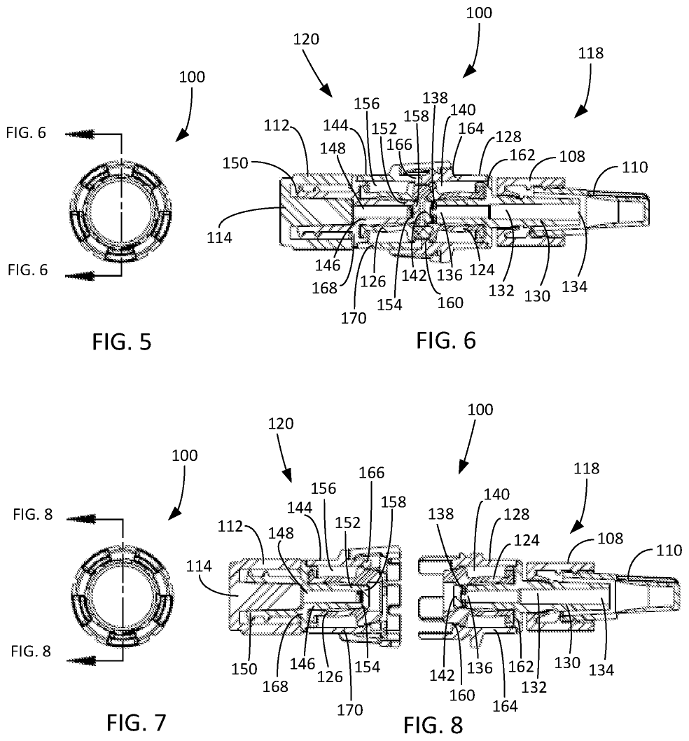

Turning to FIGS. 5-7, shown therein are end and cross-sectional views of the tubing connector system 100. The male module 118 includes a male valve housing 128 and a male module stem 130 that extends from the male valve housing 128 into the first tubing fitting 110. As illustrated in FIGS. 6 and 8, the first compression fitting 108 is configured to rotate around the male module stem 130 while drawing the first tubing fitting 110 into the central connector 106 with a threaded engagement. The male module stem 130 includes a first fluid passageway 132 that extends through the middle of the male module stem 130 from an exterior end 134 of the male module stem 130 to an interior end 136 of the male module stem 130 inside the male valve housing 128. The male module stem 130 includes one or more male module pores 138 proximate the interior end 136 of the male module stem 130. The one or more male module pores 138 are apertures in the male module stem 130 that place the first fluid passageway 132 in fluid communication with a first annular space 140 within the male valve housing 128. As depicted in FIGS. 6 and 8, the male module stem 130 includes an interior end wall 142 that forces fluid exchange between the first annular space 140 and the first fluid passageway 132 to occur through the one or more male module pores 138.

The female module 120 includes a female valve housing 144 and a female module stem 146 that extends from the female valve housing 144 to the second tubing fitting 114. As depicted in FIGS. 6 and 8, the second tubing fitting 114 is configured to fit inside the female module stem 146 and the second compression fitting 112 is configured for a threaded connection with the outside of the female module stem 146 to capture the second tubing fitting 114 in fluid communication with a second fluid passageway 148 within the female module stem 146.

The second fluid passageway 148 extends through the middle of the female module stem 146 from an exterior end 150 of the female module stem 146 to an interior end 152 of the female module stem 146 inside the female valve housing 144. The female module stem 146 includes one or more female module pores 154 proximate the interior end 152 of the female module stem 146. The one or more female module pores 154 are apertures in the female module stem 146 that place the second fluid passageway 148 in fluid communication with a second annular space 156 of the female valve housing 142. As depicted in FIGS. 6 and 8, the female module stem 146 includes an interior end wall 158 that forces fluid exchange between the second annular space 156 and the second fluid passageway 148 to occur through the one or more female module pores 154.

The first and second valve members 124, 126 are retained in the first and second annular spaces 150, 156, respectively. The male valve housing 128 includes an interior valve flange 160, a foot wall 162 and an outer wall 164 that cooperate to retain the first valve member 124. The first annular space 140 resides between the outer wall 164 and the male module stem 130. The female valve housing 144 similarly includes an interior valve flange 166, a foot wall 168 and an outer wall 170 that cooperate to retain the second valve member 126. The second annular space 156 resides between the outer wall 170 and the female module stem 146.

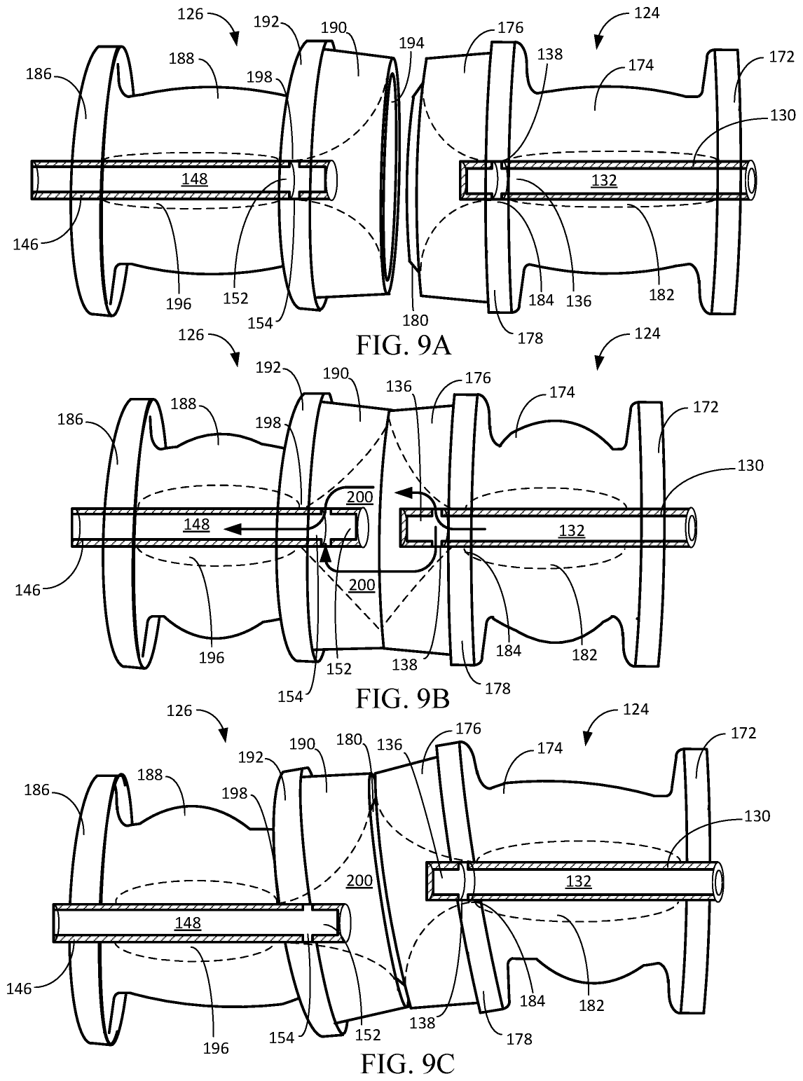

As shown in FIGS. 9A-9C, the first valve and second valve members 124, 126 are generally cylindrical and manufactured from a flexible polymer or plastic. In some embodiments, the first and second valve members 124, 126 are manufactured from an elastomeric substance such as USP class VI silicone rubber. The first valve member 124 includes a first valve foot 172, a first valve bellows 174 and a first valve head 176. The first valve head 176 has a larger outside diameter than the outside diameter of the first valve bellows 174. The first valve head 176 includes a first valve shoulder 178 and a projection 180 that extends longitudinally inward from the first valve head 176.

The first valve member 124 includes a first valve bore 182 that axially extends through the interior of the first valve member 124. During installation, the first valve member 124 is placed into the male module 118 such that the interior end 136 of the male module stem 130 is located inside the first valve bore 182. As noted in FIG. 9A, the first valve member 124 includes a first valve seal 184 formed by a narrowing of the first valve bore 182 that is radially interior to the first valve shoulder 178. The first valve seal 184 is sized and configured to cover the male module pores 138 when the first valve member 124 is in a relaxed state (as depicted in FIG. 9A).

The second valve member 126 includes a second valve foot 186, a second valve bellows 188 and a second valve head 190. The second valve head 190 has a larger outside diameter than the outside diameter of the second valve bellows 188. The second valve head 190 includes a second valve shoulder 192 and a receiver 194 that is configured accept in close tolerance the first valve projection 180 from the first valve head 176.

The second valve member 126 includes a second valve bore 196 that axially extends through the interior of the second valve member 126. During installation, the first valve member 124 is placed into the female module 120 such that the interior end 152 of the female module stem 146 is located inside the second valve bore 196. As noted in FIG. 9A, the second valve member 126 includes a second valve seal 198 formed by a narrowing of the second valve bore 198 that is radially interior to the second valve shoulder 192. The second valve seal 198 is sized and configured to cover the female module pores 154 when the second valve member 126 is in a relaxed state (as depicted in FIG. 9A).

During assembly of the tubing connector system 100, the male module 118 and female module 120 are connected (as depicted in FIG. 6) and the first and second valve members 124, 126 are engaged such that the first valve projection 180 is captured within the receiver 194 of the second valve member 126 (as depicted in FIG. 9B). When the first valve head 176 and second valve head 190 are engaged in this manner, an inter-valve flow path 200 is formed between and within the first and second valve members 124, 126.

As the male module stem 130 and female module stem 146 are approximated closer together during assembly, the first valve head 176 and second valve head 190 remain stationary. This forces the interior end 136 of the male module stem 130 to move beyond the first valve seal 184 of the first valve member 124, thereby exposing the male module pores 138. At the same time, the interior end 152 of the female module stem 146 is pressed beyond the second valve seal 198 of the second valve member 126, thereby exposing the female module pores 154. In this retracted position, the first valve bellows 174 and second valve bellows 188 are compressed and apply a spring force to maintain a sealed connection between the first valve head 176 and the second valve head 190. This ensures that fluid passing between the male module pores 138 and the female module pores 154 through the inter-valve flow path 200 is contained within the connected first and second valve members 124, 126 (as illustrated in FIG. 9B). When the male module 118 and female module 120 are separated, the first and second bellows 174, 188 push the first valve seal 184 and second valve seal 198 from a retracted position (FIG. 9B) to a deployed position (FIG. 9A) to close the male module pores 138 and female module pores 154.

Importantly, the first and second valve members 124, 126 are configured such that the first valve head 176 and second valve head 190 have a gimbaled connection to the first valve bellows 174 and second valve bellows 188, respectively. As depicted in FIG. 9C, the plug-and-socket connection between the first valve projection 180 and the receiver 194 of the second valve head 190 allows the first and second valve members 124, 126 to remain engaged without compromising the inter-valve flow path 200, even if the male module stem 130 and female module stem 146 become misaligned as the tubing connector system 100 undergoes a disconnection event. This ensures that potentially hazardous fluids are contained within the tubing connector system 100, even as the first and second valve members 124, 126 are contorted during a disconnection event.

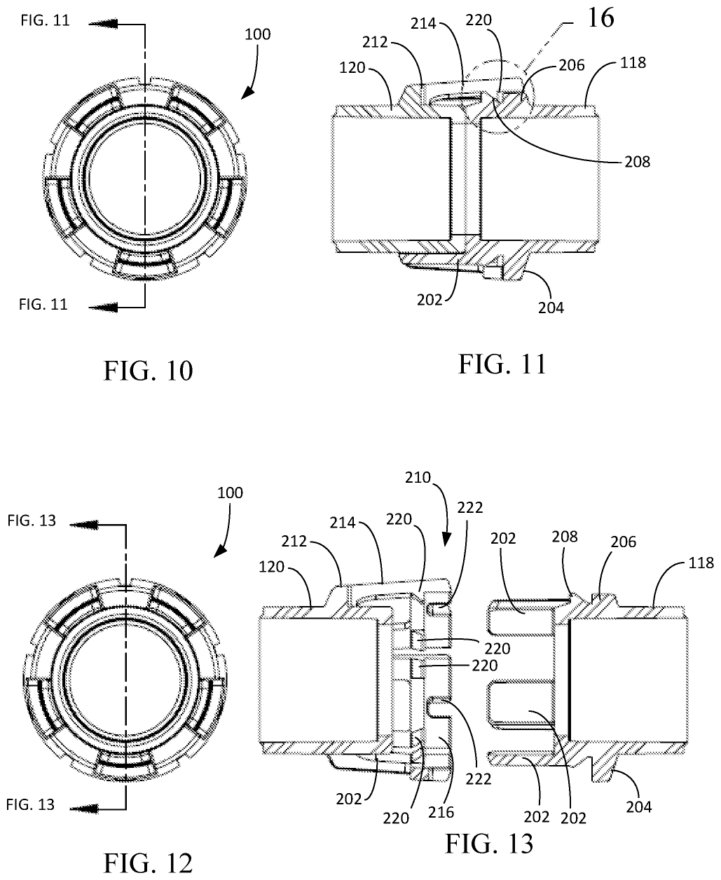

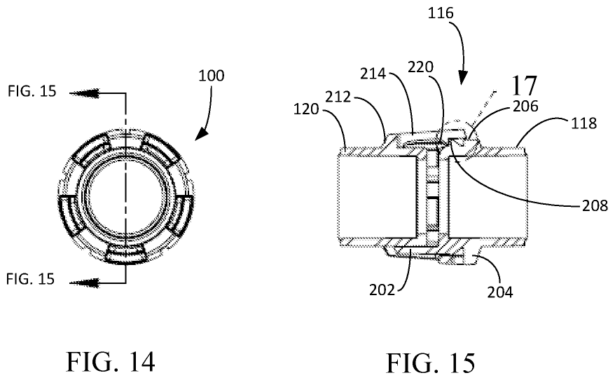

Turning to FIGS. 10-18 and also FIGS. 1-4, shown therein are various depictions of the breakaway assembly 116, and in particular the interconnecting exterior elements of the male module 108 and female module 120 (the detailed interior elements of the male module 108 and female module 120 have been removed for clarity). Generally, the male module 118 and female module 120 include a plurality of locking engagement features that cooperate to provide a limited, longitudinal linear range of motion as the male module 118 and female module 120 are locked together during assembly. In addition to limiting rotational, bending or radial movements between the male module 118 and female module 120, these features also prevent the male module 118 and female module 120 from being reconnected after a disconnection event. This prevents the reuse of the tubing connector system 100 once the male module 118 and female module 120 have been disconnected in a non-sterile environment.

The male module 118 includes a plurality of stabilizers 202 that project longitudinally inward toward the female module 120. Each stabilizer 202 is generally configured as a finger or tab with a radial curvature that matches the cylindrical form of the male module 118. In exemplary embodiments, the stabilizers 202 are equally distributed around the circumference of the male module 118. The male module 118 also includes a plurality of alignment tabs 204 that extend radially outward from an abutment ring 206 that circumferentially extends around the outside of the male module 118. The male module 118 further includes a locking ring 208 that circumferentially extends around the male module 118 on the interior side of the abutment ring 206. As best depicted in FIG. 4, the stabilizers 202 can be connected to, and extend from, the locking ring 208.

The female module 120 includes a locking collar 210 that circumferentially extends around the interior end of the female module 120. The locking collar 210 is attached to the outside of the female module 120 by a plurality of collar mounts 212. The locking collar 210 includes a plurality of locking tabs 214 that extend inward toward the male module 118. Each locking tab 214 is generally configured as a U-shaped member in which two proximal ends are affixed to adjacent collar mounts 212 to support a cross member 216 in a cantilevered manner. In this way, each locking tab 214 presents a stabilizer recess 218 between the adjacent collar mounts 212 to which the locking tab 214 is attached.

As best illustrated in FIGS. 11 and 13, the locking tabs 214 are attached to the collar mounts 212 with a reinforced, curved interior that limits the outward flex of the cantilevered locking collar 210. Furthermore, because the locking collar 210 is constructed as a circular member, the distal, free end of the locking collar exhibits a “hoop strength” that further resists an outward radial flex. In this way, the locking collar 210 exhibits a spring-force resistance to an application of force in an outward radial direction.

The cross member 216 of each locking tab 214 includes a plurality of teeth 220 that project radially inward from the cross member 216. As depicted, each cross member 216 includes a pair of teeth 220 disposed in a spaced apart relationship on the outside ends of the cross member 216. The teeth 220 are spaced back from the distal end of the cross member 216. The cross member 216 also includes an alignment tab recess 222 on the inward side of the middle of the cross member 216. Each alignment tab recess 222 is configured to accommodate a corresponding one of five alignment tabs 204 extending radially outward from the abutment ring 206 of the male module 118. The locking tabs 214 and teeth 220 are cylindrically contoured to match the circular shape of the corresponding locking features of the male module 118.

As illustrated, the female module 120 includes five locking tabs 214 extending from the locking collar 210, which produces five stabilizer recesses 218 to accommodate five stabilizers 202 from the male module 118. It will be appreciated that these locking features can be equally spaced and distributed around the circumference of the male and female modules 118, 120. Although five locking tabs 214, five stabilizers 202 and five alignment tabs 204 are depicted in illustrated embodiments, it will be appreciated that greater or fewer numbers of these features are contemplated as within the scope of these embodiments. For example it may be desirable to include 3, 4, 5, 6, 7, 8, 9, 10 or 11 of each of the locking tabs 214, stabilizers 202 and alignment tabs 204. For some applications, an odd number of each of these locking features is preferred over an even number of locking features.

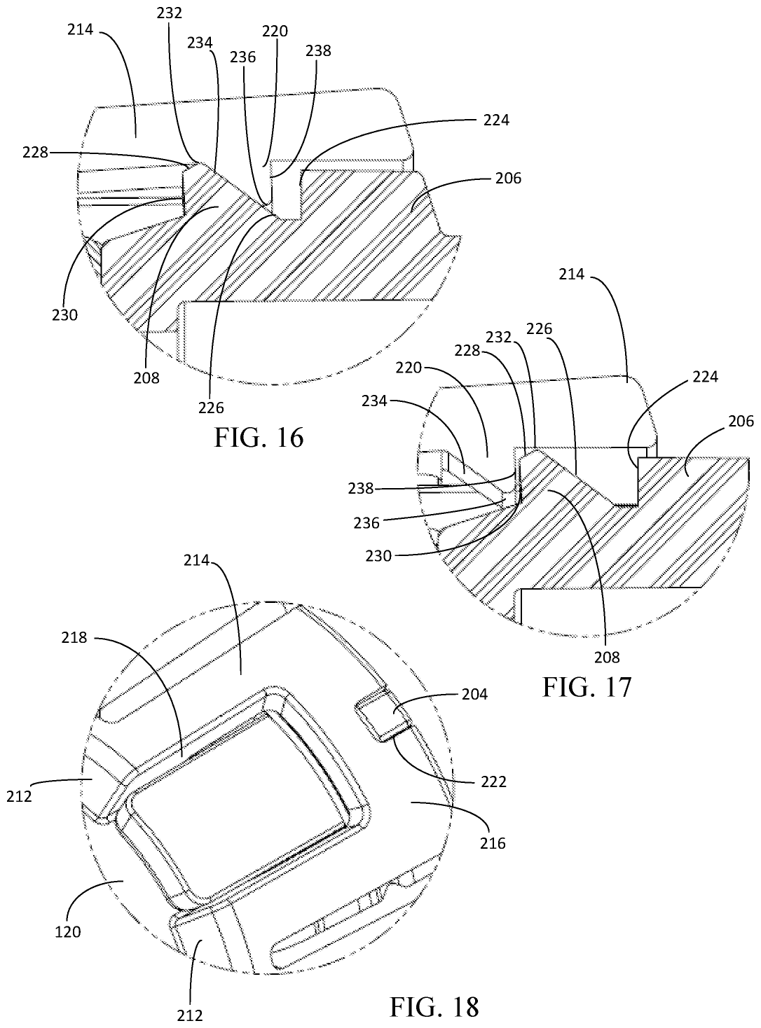

As best illustrated in the close-up views in FIGS. 16-18, the abutment ring 206 includes an abutment face 224 that extends radially outward in a plane that is substantially orthogonal to the central longitudinal axis extending through the tubing connector system 100. The locking ring 208 includes a sloped locking face 226, a sloped release face 228 and a blocking face 230. With reference to a plane extending orthogonal to the central longitudinal axis through the peak of the locking ring 208, the locking face 226 extends outward and radially inward at an angle of between 10 and 80 degrees. As depicted, the locking face 226 extends longitudinally outward away from the female module 120 and radially inward at approximately 45 degrees. With reference to the same orthogonal plane, the release face 228 slopes radially inward and longitudinally toward the female module 120 at approximately 45 degrees. In other embodiments, the release face 228 can be configured with an angle between 10 and 80 degrees. The locking face 226 meets the release face 228 at a rounded peak 232. The blocking face 230 extends radially outward in a plane that is substantially orthogonal to the central longitudinal axis. In some embodiments, the longitudinal length of the locking ring 208 is approximately the same as the longitudinal length of each of the alignment tabs 204.

Each tooth 220 includes a sloped face 234 with an angle that is substantially congruent to the angle of the locking face 226 (as illustrated in FIG. 16). Each tooth 220 has a rounded tooth apex 236 that joins the sloped face 234 with a preventer face 238. In this way, the sloped face 234 and the preventer face 238 are oriented at an angle of between 10 and 80 degrees, with an angle of about 45 degrees in FIG. 16.

When the male module 118 and female module 120 are connected, each stabilizer 202 is captured within a corresponding one of the stabilizer recesses 218. Each alignment tab 204 is captured within a corresponding one of the alignment tab recesses 222. Each tooth 220 is engaged in a locked position in which the sloped face 234 is engaged in contact with the sloped locking face 226 of the locking ring 208 (as depicted in FIGS. 1, 2, 6, 11, 16 and 18). Further approximation of the male and female modules 118, 120 is prevented by contact between the preventer face 238 and the abutment face 224. In this position, the captured stabilizers 202 and captured alignment tabs 204 prevent the rotation, bending or further approximation of the male and female modules 118, 120. The only permitted motion—the linear separation of the male and female modules 118, 120—is limited by the engagement of the teeth 220 on the locking ring 208.

Attempts to separate the male and female modules 118, 120 by pulling them in opposite directions along the central longitudinal axis causes the locking collar 210 to flex radially outward as the teeth 220 are pulled up the locking face 226 of the locking ring 208. The rigidity of the locking collar 210 resists this deformation until the tension between the male and female modules 118, 120 causes the tooth apex 236 to reach the peak 232 of the locking ring 208 between the locking face 226 and the release face 228. The peak 232 and tooth apex 236 are designed so that the relative movement between the male module 118 and female module 120 does not stall when the peak 232 is in contact with the tooth apex 236. When the tooth apex 234 is pulled into contact with the peak 232, the inward pressure exerted by the locking collar 210 to the teeth 220 on the inward slope of the release face 228 causes the male and female modules 118, 120 to rapidly spring apart. This ensures that once the male and female 118, 120 modules have been pulled apart by a “threshold longitudinal separation distance” in response to a “threshold separation force,” which would potentially compromise the seal provided by the valve assembly 122, the male and female modules 118, 120 quickly separate and the first and second valve members 124, 126 return to the deployed state to prevent leakage or contamination through the male and female module pores 138, 154. Thus, in exemplary embodiments, the longitudinal distance of travel for the first valve seal 184 and second valve seal 198 (the “valve travel distance”) is less than the threshold longitudinal separation distance to ensure that the valve assembly 122 deploys to the closed position before the male and female modules 118, 120 separate.

Importantly, the threshold longitudinal separation distance is optimally the same distance as the longitudinal length of the alignment tabs 204 and alignment tab recesses 222. This prevents the male and female modules 118, 120 from rotating with respect to one another until the two components have been unlocked and disengaged. The threshold separation force required to separate the male and female modules 118, 120 can be adjusted by altering the geometry of the mating components of the locking ring 208 and teeth 220. Increasing the slope of the locking face 226 and sloped face 234 will increase the amount of tensile force required to separate the male and female modules 118, 120. Similarly, the threshold longitudinal separation distance can be adjusted by increasing or decreasing the longitudinal distance of one or both of the locking face 226 and the sloped face 234.

Once the male and female modules 118, 120 have been separated (as depicted in FIGS. 3, 4, 8, 13, 15 and 17), the geometry of the locking ring 208 and teeth 220 prevent the reconnection of the central connector 106. As best illustrated in FIG. 17, the preventer face 238 and blocking face 230 are each oriented such that pressing the male and female modules 118, 120 together simply causes the preventer face 238 of the teeth 220 to abut the blocking face 230. The blocking face 230 prevents the teeth 220 from being pressed up and over the locking ring 208. Significantly, the teeth 220 are positioned proximal to the distal end of the locking tabs 214 by a distance that is sufficiently large to cover the abutment ring 206 when the central connector 106 is locked (as shown in FIG. 16) or to cover the locking face 226 of the locking ring 208 when the central connector 106 has been separated (as shown in FIG. 17). This prevents a patient or caregiver from using a tool to pry under the locking collar 210 in an attempt to lift the locking collar 210 and teeth 220 to separate or reconnect the male and female modules 118, 120 of the central connector 106.

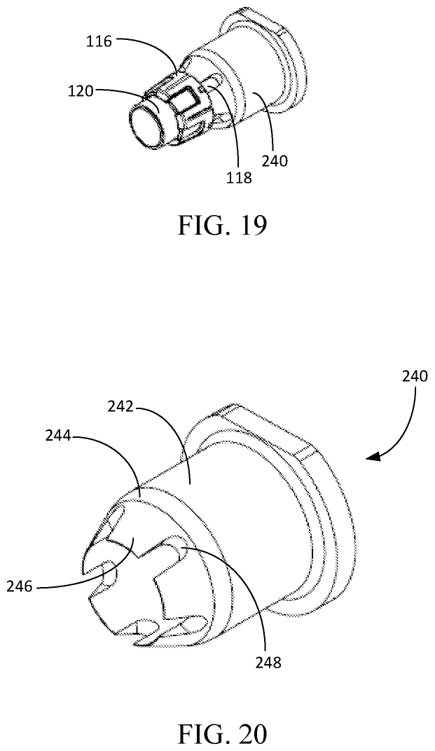

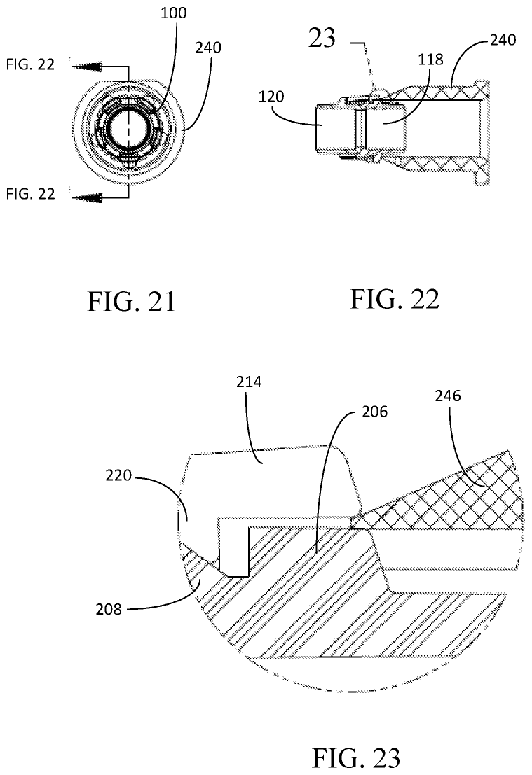

During assembly, the male module 118 is engaged with the female module 120 using an assembly tool 240, depicted in FIGS. 19-23. The assembly tool 240 has a cylindrical body 242 with a conical nose 244 that includes a series of wedges 246 and reliefs 248. The reliefs 248 are sized and arranged to accommodate the alignment tabs 204 as the male module 118 passes through the assembly tool 240. The assembly tool 240 is inserted over the male module 118 in a manner in which the wedges 246 are placed between the distal end of the locking tabs 214 of the female module 120 and the abutment ring 206 of the male module 118. The assembly tool 240 is pressed into the female module 120 to outwardly splay the locking collar 210 by an amount necessary to permit the peak 232 of the locking ring 208 to pass within the apex 236 of each of the inwardly directed teeth 220. A separate tool can be used to press the male module 118 into the female module 120. Once the locking ring 208 has passed within the out-splayed teeth 220, the assembly tool 240 can be withdrawn while holding the position of the male module 118, thereby allowing the spring-loaded locking collar 210 to press the teeth 220 into locked engagement with the locking ring 208. FIG. 23 depicts the withdrawal of the assembly tool 240 to lower the teeth 220 of the locking collar 210 into the locking ring 208.

It is clear that the present invention is well adapted to carry out its objectives and attain the ends and advantages mentioned above as well as those inherent therein. While presently preferred embodiments of the invention have been described in varying detail for purposes of disclosure, it will be understood that numerous changes may be made which will readily suggest themselves to those skilled in the art and which are encompassed within the spirit of the invention disclosed herein.

It is claimed:

1. A tubing connector system for use in connecting two pieces of medical tubing, the tubing connector system comprising:
a first tubing adapter;
a second tubing adapter; and
a central connector positioned between the first adapter and the second tubing adapter,
wherein the central connector comprises:
a male module;
a female module;
a valve assembly, wherein the valve assembly comprises:
a first valve member retained within the male module; and
a second valve member retained within the female module; and
wherein the male module and female module are configured to prevent reconnection once the male module and female module have been separated.

2. The tubing connector system of claim 1, wherein the male module comprises:
a locking ring;
an abutment ring;
a plurality of alignment tabs extending radially outward from the abutment ring; and
a plurality of stabilizers.

3. The tubing connector system of claim 2, wherein the female module comprises a locking collar.

4. The tubing connector system of claim 3, wherein the locking collar comprises:
a plurality of locking tabs that engage the locking ring;
a plurality of alignment tab recesses, wherein each of the plurality of alignment tab recesses is configured to accept a corresponding one of the plurality of alignment tabs;
a plurality of locking tabs that engage the locking ring; and
a plurality of stabilizer recesses, wherein each of the plurality of stabilizer recesses is configured to accept a corresponding one of the plurality of stabilizers.

5. The tubing connector system of claim 1, wherein the first valve member comprises:
a first valve foot;
a first valve head; and
a first valve bellows between the first valve foot and the first valve head.

6. The tubing connector system of claim 5, wherein the first valve member further comprises a first valve projection extending from the first valve head.

7. The tubing connector system of claim 6, wherein the second valve member comprises:
a second valve foot;
a second valve head; and
a second valve bellows between the second valve foot and the second valve head.

8. The tubing connector system of claim 7, wherein the second valve member further comprises a receiver within the second valve head and wherein the receiver is configured to receive the projection of the first valve member.

9. The tubing connector system of claim 1, wherein the male module comprises a male module stem that includes one or more male module pores.

10. The tubing connector system of claim 9, wherein the female module comprises a female module stem that includes one or more female module pores.

11. The tubing connector system of claim 10, wherein the first valve member selectively seals the one or more male module pores on the male module stem and wherein the second valve member selectively seals the one or more female module pores on the female module stem.

12. A tubing connector system configured to connect first and second pieces of medical tubing, the tubing connector system comprising:
a male module;
a female module, wherein the female module is retained in connection to the male module with a plurality of locking mechanisms; and
wherein the plurality of locking mechanisms are configured to prevent the male module and female module from being reconnected after the male module has been separated from the female module.

13. The tubing connector system of claim 12, wherein the plurality of locking mechanisms are selected from the group consisting of stabilizers and stabilizer recesses configured to accept the stabilizers, alignment tabs and align tab recesses configured to accept the alignment tabs, and a locking ring and locking tabs configured to engage with the locking ring.

14. The tubing connector system of claim 12, wherein the female module comprises:
a locking collar, wherein the locking collar comprises:
a plurality of collar mounts; and
a plurality of locking tabs, wherein each of the plurality of locking tabs is supported by a corresponding pair of adjacent collar mounts in a cantilevered configuration, wherein each of the plurality of locking tabs comprises:
a crossmember; and
a stabilizer recess; and
a plurality of teeth extending radially inward from the crossmember.

15. The tubing connector system of claim 14, wherein the male module comprises:
a locking ring, wherein the locking ring is configured to engage the plurality of teeth of the locking collar; and
a plurality of stabilizers extending from the locking ring, wherein each of the plurality of stabilizers is configured to extend longitudinally into the stabilizer recess of a corresponding one of the locking tabs.

16. The tubing connector system of claim 15, wherein the locking ring comprises:
a locking face;
a release face;
a peak between the locking face and the release face; and
a blocking face.

17. The tubing connector system of claim 16, wherein each of the plurality of teeth comprises:
a sloped face;
a preventer face; and
a tooth apex between the sloped face and the preventer face.

18. The tubing connector system of claim 17, wherein the locking collar is secured to the locking ring when the sloped face of each of the plurality of teeth is in contact with the locking face of the locking ring.

19. The tubing connector system of claim 17, wherein the locking collar and locking ring are forced to separate when the tooth apex passes from the locking face over the peak onto the release face.

20. The tubing connector system of claim 17, wherein the preventer face and the blocking face prevent the locking collar from being reattached to the locking ring once the tooth apex passes from the locking face over the peak onto the release face.

21. A tubing connector system configured to connect a first piece of medical tubing to a second piece of medical tubing, the tubing connector system comprising:
a male module;
a female module; and
means for releasably locking the male module to the female module, wherein the means for releasably locking the male module to the female module also prevent the male module from being reattached to the female module.
